(19) United States
(12) Patent Application Publication
(10) Pub. No.: US 2017/0266223 A1
(43) Pub. Date: Sep. 21, 2017

(54) NOVEL METHODS OF PREPARING BIOMIMETIC PROTEOGLYCANS

(71) Applicant: DREXEL UNIVERSITY, Philadelphia, PA (US)

(72) Inventors: Michele MARCOLONGO, Aston, PA (US); Katsiaryna PRUDNIKOVA, Huntingdon Valley, PA (US); Lynn S. PENN, Philadelphia, PA (US)

(21) Appl. No.: 15/505,262

(22) PCT Filed: Aug. 25, 2015
(86) PCT No.: PCT/US2015/046768

§ 371 Date: Feb. 21, 2017

Related U.S. Application Data
(60) Provisional application No. 62/041,980, filed on Aug. 26, 2014.

(51) Int. Cl. A61K 31/78; C08F 120/02; A61K 45/06

(52) U.S. Cl. CPC A61K 31/78; A61K 45/06; C08F 120/02

(57) ABSTRACT

The present invention novel methods of preparing biomimetic proteoglycans, such as bottle-brush, chondroitin sulfate-containing, biomimetic proteoglycans. In certain embodiments, the methods of the invention comprise contacting a core polymer comprising at least one acyl chloride group with a GAG comprising a terminal primary amine.

### CROSS-REFERENCE TO RELATED APPLICATIONS

The present application claims priority under 35 U.S.C. §119(e) to U.S. Provisional Patent Application No. 62/041,980, filed Aug. 26, 2014, which application is incorporated herein by reference in its entirety.

### BACKGROUND OF THE INVENTION

Proteoglycans are heavily glycosylated proteins present in the connective tissue. The basic proteoglycan unit consists of a “core protein” with one or more covalently attached glycosaminoglycan (GAG) chain(s). The point of attachment on the core protein is a serine residue, to which the GAG chain is joined through a tetrasaccharide linker. The serine residue is generally in the sequence -Ser-Gly-X-Gly- (where X can be any amino acid residue except proline), but not every protein with this sequence has an attached glycosaminoglycan. The GAG chains are long, linear carbohydrate polymers that are negatively charged under physiological conditions, due to the presence of sulfate and uronic acid groups.

Proteoglycans are major components of the animal extracellular matrix, forming large complexes with other proteoglycans, hyaluronan and/or fibrous matrix proteins (e.g., collagen). The functions of proteoglycans can be attributed to the protein core and/or the GAG chain. Proteoglycans serve as lubricants, bind cations (e.g., sodium, potassium and calcium) and water, and regulate the movement of molecules through the matrix. Further, proteoglycans can affect the activity and stability of proteins and signaling molecules within the matrix.

Proteoglycans can be categorized according to their size and/or the nature of their GAG chains. Types of GAG chains include: chondroitin sulfate/dermatan sulfate, which is present in the proteoglycans decorin (36 kDa), biglycan (38 kDa) and versican (260-370 kDa); heparan sulfate/chondroitin sulfate, which is present in the proteoglycans testican (44 kDa) and perlecan (400-470 kDa); chondroitin sulfate, which is present in the proteoglycans bikunin (25 kDa), neurocan (136 kDa) and aggrecan (220 kDa; the major proteoglycan in cartilage); and keratan sulfate, which is present in the proteoglycans fibromodulin (42 kDa) and lumican (38 kDa).

In particular, chondroitin sulfate proteoglycans are structural components of a various human tissues (e.g., cartilage), and also play key roles in neural development and glial scar formation. They are involved in cell processes such as cell adhesion, cell growth, receptor binding, cell migration, and interaction with other extracellular matrix constituents. They also interact with laminin, fibronectin, tenascin, and collagen. Importantly, chondroitin sulfate proteoglycans inhibit axon regeneration after spinal cord injury. They contribute to glial scar formation post injury, acting as a barrier against new axons growing into the injury site.

Developmental approaches exist to surgically repair or replace degenerated tissues, including degenerated tissue, which includes components of aggregated aggrecan (e.g., protein core, condroitin sulfate, keratan sulfate and HA).

U.S. Patent Application Publication No. US 2013/0052155, which is incorporated herein in its entirety by reference, discloses a novel biosynthetic chondroitin sulfate-containing proteoglycan that is enzymatically resistant and can be used to replace proteoglycans in natural tissues. Interestingly, the molecular design advances the survival of the molecule in vivo, while maintaining molecular function.

There is a need in the art for improved methods of synthesizing biosynthetic proteoglycans. Such methods should allow for the efficient and flexible synthesis of the proteoglycans that can be used in restoring and/or replacing damaged or degenerated tissues and/or bone, including intervertebral discs. The present invention satisfies this need.

### BRIEF SUMMARY OF THE INVENTION

The invention provides a method of preparing a biomimetic proteoglycan. The invention further provides a biomimetic proteoglycan prepared according to any of the method s of the invention, wherein the proteoglycan is optionally in a composition. The invention further provides a method of treating a disease, disorder, or condition associated with a soft tissue in a mammal in need thereof.

In certain embodiments, the method comprises contacting a core polymer comprising at least one acyl chloride group and a GAG comprising a terminal primary amine, thereby forming a biomimetic proteoglycan.

In certain embodiments, the GAG is selected from the group consisting of hyaluronic acid, chondroitin, chondroitin sulfate, heparin, heparin sulfate, dermatan, dermatan sulfate, laminin, keratan sulfate, chitin, chitosan, acetyl-glucosamine, oligosaccharides, and any combinations thereof. In other embodiments, the GAG comprises chondroitin sulfate.

In certain embodiments, the core polymer is in an organic solution, wherein the organic solution is not fully soluble in water or an aqueous solution. In other embodiments, the core polymer is in an organic solution comprising ethyl acetate, dioxane, tetrahydrofuran, dichloroethane, dichloromethane, cyclohexane, or any mixtures thereof. In yet other embodiments, the GAG is in an aqueous solution. In yet other embodiments, the GAG is in a buffered aqueous solution. In yet other embodiments, the GAG is in an aqueous solution of pH ranging from about 5.5 to about 9.4.

In certain embodiments, the polymer core comprises acyl chloride-containing derivatives of poly(acrylic acid), poly(methacrylic acid), poly(glutamic acid) or poly(aspartic acid), or copolymers, mixtures, and combinations thereof.

In certain embodiments, at least a portion of the acyl chloride groups in the core polymer does not react with the terminal primary amines of the GAGs.

In certain embodiments, the method further comprises reacting the unreacted acyl chloride groups with a nucleophile or base. In other embodiments, all or about all of the acyl chloride groups in the core polymer react with the terminal amines of the GAGs.

In certain embodiments, the biomimetic proteoglycan is resistant to enzymatic breakdown in a mammalian in vivo environment. In other embodiments, the biomimetic proteoglycan has a shape selected from the group consisting of cyclic, linear, branched, star-shaped, comb, graft, bottlebrush, dendritic, mushroom, and any combinations thereof. In yet other embodiments, the biomimetic proteoglycan mimics a natural proteoglycan selected from the group consisting of aggrecan, betaglycan, decorin, perlecan, serglycin, syndecan-1, biglycan, fibromodulin, lumican, versican, neurocan, brevican, and any combinations thereof. In yet other embodiments, the soft tissue is selected from the group consisting of intervertebral disc, skin, heart valve, articular cartilage, cartilage, meniscus, fatty tissue, craniofacial, ocular, tendon, ligament, fascia, fibrous tissue, urethra, bone, synovial membrane, muscle, nerves, blood vessel, and any combinations thereof.

In certain embodiments, the composition further comprises at least one biologically active molecule selected from the group consisting of a growth factor, cytokine, antibiotic, protein, anti-inflammatory agent, and analgesic.

In certain embodiments, the method comprises administering to the mammal a therapeutically effective amount of any of the compositions of the invention. In other embodiments, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph illustrating experimental results relating to the conjugation of chondroitin sulfate (CS) to poly(acryloyl chloride) using the methods of the invention.

FIG. 2 and FIG. 3 are graphs illustrating osmotic pressure in solution and water uptake of poly(acryloyl chloride)-chondroitin sulfate molecule, respectively.

### DETAILED DESCRIPTION OF THE INVENTION

The invention relates to novel methods of preparing biomimetic glycosaminoglycan (“GAG” hereinafter)-containing proteoglycans. The methods of the invention comprise coupling a GAG to a core polymer. In certain embodiments, the GAG comprises a terminal primary amine, which is used to covalently couple the GAG to the core polymer. In other embodiments, the core polymer comprises one or more acyl chloride groups, which react with the terminal primary amine of the GAG to form an amide group, which links the GAG to the core structure. In yet other embodiments, the biomimetic proteoglycan comprises a bottle brush structure. In yet other embodiments, the biomimetic proteoglycan exhibits one or more characteristics of natural chondroitin sulfate bristles.

In certain embodiments, the methods of the invention allow for controlled organization of glycosaminoglycans onto various polymeric backbones, wherein the properties of the resulting biomimetic proteoglycan is tuned as to be used in treating a disease, disorder, or condition associated with dysfunctional proteoglycan. In other embodiments, the biomimetic proteoglycans prepared according to the methods of the invention are an improvement over their corresponding natural counterparts. In yet other embodiments, the biomimetic proteoglycans comprise enzymatically resistant core.

In certain embodiments, the biomimetic proteoglycans prepared according to the methods of the invention have controllable enzymatic liability, at least in part based on the choice of core polymer. In other embodiments, the biomimetic proteoglycans prepared according to the methods of the invention are large enough to resist migration out of the desired site of administration. In yet other embodiments, the biomimetic proteoglycans prepared according to the methods of the invention enhance and/or do not interfere with cellular metabolic activity that depends on convection for the large molecule metabolites.

In certain embodiments, the biomimetic proteoglycans bind to collagen with binding affinities on the same order as natural aggrecan.

The present invention allows for the preparation of compositions useful for treating diseases, disorders, or conditions associated with soft tissue defects and disorders, where administration of a biomimetic proteoglycan to the soft tissue site results in functional restoration of the soft tissue, in whole or in part.

The present invention can be used in conjunction with any known or heretofore unknown method of treating a disease or condition in a mammal. Preferably, the mammal is a human.

In certain embodiments, the invention includes a kit comprising a biomimetic proteoglycan, an introducer needle, and a delivery device for administering the biomimetic proteoglycan. The biomimetic proteoglycan may be administered as a solution or dry. In some instances, the kit further comprises an instruction manual.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, “about” when referring to a measurable value such as an amount, a temporal duration, and the like is meant to encompass variations of ±20% or ±10%, more preferably ±5%, even more preferably ±1%, and still more preferably ±0.1% from the specified value, as such variations are appropriate to perform the disclosed methods.

The term “biologically compatible carrier” or “biologically compatible medium” refers to reagents, cells, compounds, materials, compositions, and/or dosage formulations that are suitable for use in contact with the tissues of human beings and animals without excessive toxicity, irritation, allergic response, or other complication commensurate with a reasonable benefit/risk ratio.

As used herein, the term “bone condition (or injury or disease)” refers to disorders or diseases of the bone including, but not limited to, acute, chronic, metabolic and non-metabolic conditions of the bone. The term encompasses conditions caused by disease, trauma or failure of the tissue to develop normally. Examples of bone conditions include, but are not limited, a bone fracture, a bone/spinal deformation, osteosarcoma, myeloma, bone dysplasia, scoliosis, osteoporosis, osteomalacia, rickets, fibrous osteitis, renal bone dystrophy, and Paget's disease of bone.

As used herein, the term “CS” refers to chondroitin sulfate.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “ECM” refers to extracellular matrix.

“Functional restoration of a tissue,” as that phrase is used herein, refers to the restoration of at least one function to a tissue, which function has been lost by the tissue as a result of a disorder or defect.

The terms “glycosaminoglycan” and “GAG”, as used interchangeably herein, refer to a macromolecule comprising a carbohydrate. The GAGs for use in the present invention may vary in size and be either sulfated or non-sulfated. The GAGs which may be used in the methods of the invention include, but are not limited to, hyaluronic acid, chondroitin, chondroitin sulfates (e.g., chondroitin 6-sulfate and chondroitin 4-sulfate), heparin, heparin sulfate, dermatan, dermatan sulfate, laminin, keratan sulfate, chitin, chitosan, acetyl-glucosamine, and the like.

By “growth factors” is intended the following specific factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), and bone morphogenetic protein at concentrations of between picogram/ml to milligram/ml levels.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Metabolically absorbable” refers herein to any chemicals or materials that are (a) safely accepted within the body with no adverse reactions, and (b) completely eliminated from the body over time through natural pathways or internal consumption. “Metabolically acceptable” refers to any chemicals or materials that are safely accepted within the body with no adverse reactions or harmful effects.

As used herein, “mimics natural proteoglycan” means mimicking the structure and function of natural proteoglycan.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

As used herein, a “polymer backbone” refers to the moiety or structure for which GAGs, such as chondroitin sulfate, can attach to form a biomimetic proteoglycan. In some instances, the polymer backbone is considered the core structure, core portion, polymer core, or protein portion of the biomimetic proteoglycan. In some instances, the polymer backbone can be a synthetic polymer, protein, peptide, nucleic acid, carbohydrate or combinations thereof.

As used herein, the term “SBB” refers to sodium borate buffer.

As used herein, “soft tissue” refers to a tissue that connects, supports, or surrounds other structures and organs of the body. For example, soft tissue includes but is not limited to disc, collagen, meniscus, tendon, ligament, fascia, fibrous tissue, fat, synovial membrane, other connective tissue, muscle, nerves, blood vessel, and the like.

For the purposes of the present invention, a soft tissue defect or disorder includes but is not limited to degeneration or damage to skin, heart valves, articular cartilage, cartilage, meniscus, fatty tissue, craniofacial, ocular, disc, and the like. The compositions prepared using the methods of the invention are also useful for repair, restoration or augmentation of soft tissue defects or contour abnormalities. Thus, the invention should be read at all times to include repair of defects in any soft tissue in the body, as the term soft tissue is defined herein. While the precise compositions used and the methods of administration of the materials of the invention may vary from tissue to tissue, the skilled artisan will know, based on the disclosure provided herein, how to adapt the disclosure to repair of any soft tissue and/or bone, to the extent that such adaption has not been disclosed in detail herein.

As used herein, a “therapeutically effective amount” is the amount of material sufficient to provide a beneficial effect to the subject to which the material is administered.

“Treating (or treatment of)” refers to ameliorating the effects of, or delaying, halting or reversing the progress of, or delaying or preventing the onset of, a disease or degenerative condition.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### DESCRIPTION

The invention relates to novel methods of preparing biomimetic proteoglycans, such as biosynthetic aggrecans. The methods of the invention comprise coupling a GAG to a core polymer. In certain embodiments, the GAG comprises a terminal primary amine, which is used to covalently link the GAG to the core polymer. In other embodiments, the core polymer comprises one or more acyl chloride groups, which react with the terminal primary amine of the GAG to form an amide group, which covalently links the GAG to the core structure. In yet other embodiments, the GAG comprises chondroitin sulfate. In yet other embodiments, the biomimetic proteoglycan comprises a bottle brush structure. In yet other embodiments, the biomimetic proteoglycan exhibits characteristics of natural chondroitin sulfate bristles.

### Methods

The methods of the invention allow for the tunable and efficient preparation of biomimetic proteoglycans. The methods comprise coupling a glycosaminoglycan (GAG) to a core polymer. Based on the disclosure herein, a skilled artisan would understand that the biomimetic proteoglycan of the invention can be engineered to encompass any type of glycosaminoglycan and combinations thereof with any type of core protein or core polymer. In certain embodiments, the biomimetic proteoglycan functions in a similar fashion to a natural proteoglycan that otherwise can be isolated from an animal or a cell. For example, the biomimetic proteoglycan is spheroidal (e.g., bottle-brush-like spatial presentation or configuration) and functionally able to maintain high levels of hydration and exhibits sufficient mechanical properties. In other embodiments, the biomimetic proteoglycan functions in a distinct and advantageous fashion to a natural proteoglycan that otherwise can be isolated from an animal or a cell.

In certain embodiments, the methods of the invention comprise contacting a core polymer comprising at least one acyl chloride group and a GAG comprising a terminal amino group. In other embodiments, the core polymer is in an organic solution, wherein the organic solution is not fully miscible with water or an aqueous solution. In yet other embodiments, the core polymer is in a solution comprising ethyl acetate, dioxane, tetrahydrofuran, dichloroethane, dichloromethane, or cyclohexane. In yet other embodiments, the GAG is in an aqueous solution. In yet other embodiments, the GAG is in a buffered aqueous solution. In yet other embodiments, the GAG is in an aqueous solution ranging in pH from about 5.5 to about 9.4, from about 5.5 to about 10, from about 8.0 to about 9.4, or from about 8.0 to about 10.0. In yet other embodiments, the GAG is in an aqueous solution of pH about 9.4.

The ratio of the core polymer comprising at least one acyl chloride and the GAG comprising a terminal amino group may be varied as a way to control the final number of GAGs attached to the core polymer. In certain embodiments, all or about all of the acyl chloride groups on the core polymer react with the GAGs. In other embodiments, a fraction of the acyl chloride groups on the core polymer reacts with the GAGs. In the case wherein a fraction of the acyl chloride groups on the core polymer react with the GAGs, the unreacted fraction of the acyl chloride groups on the core polymer may be reacted with additional nucleophilic reagents, such as but not limited to amines, thiols, alcohols, amino acids, peptides, small molecules, lipids and the like. Alternatively, the unreacted fraction of the acyl chloride groups on the core polymer may be hydrolyzed by containing the core polymer with a base, such as but not limited to a metal hydroxide, whereby a carboxylic group is formed on the core polymer. The ratio of final carboxylic acid groups and units of GAG attached to the copre polymer may be tuned to generate a biomimetic proteoglycan of desired physico-chemical properties.

In certain embodiments, the GAG comprises a terminal primary amine handle that is used to attach the GAG to the core structure through an amide bond. Non-limiting examples of GAGs include hyaluronic acid, chondroitin, chondroitin sulfates (e.g., chondroitin 6-sulfate and chondroitin 4-sulfate), heparin, heparan sulfate, dermatan, dermatan sulfate, laminin, keratan sulfate, chitin, chitosan, acetyl-glucosamine, and the like.

In certain embodiments, the biomimetic proteoglycan can encompass any combination of glycosaminoglycans wherein each glycosaminoglycan can vary in length. Similarly, varying lengths of the polymer can be used in the construction of the biomimetic proteoglycan. Without wishing to be bound by any particular theory, glycosaminoglycan variations include but are not limited to varying length, sulfation pattern, molecular weight, chemical composition, and the like. These variations can affect the conformation, molecular weight, hydrating, mechanical and cell signaling functions of the biomimetic proteoglycan.

In certain embodiments, any polymer core comprising acyl chloride groups can be used for attachment of the desired glycosaminoglycan. Polymers that may be used as the core portion of the biomimetic proteoglycan include, but are not limited to, acyl chloride-containing derivatives of poly(acrylic acid), poly(methacrylic acid), copolymers of acrylic acid and methacrylic acid, poly(glutamic acid), poly(aspartic acid), copolymers, mixtures, derivatives and combinations thereof. As such, the carboxylic acid groups in carboxylic acid-containing polymers may be at least partially converted to the corresponding acyl chlorides and then used within the methods of the invention. Linear and branched polymers may be used in the biomimetic proteoglycan of the present invention. The backbone is also useful for providing supramolecular structure to the resulting biomimetic proteoglycan and/or components thereof.

In certain embodiments, the glycosaminoglycan is grafted to a backbone polymer with a predetermined number of attachment sites. Accordingly, the density of glycosaminoglycan to polymer can be adjusted to correspond to the particular use of the biomimetic proteoglycan.

The biomimetic proteoglycan can also be designed to have a particular shape. For example, different types of polymeric backbones can be used to generate a biomimetic proteoglycan that may take on a number of configurations, which may be selected, for example, from cyclic, linear and branched configurations, among others. Branched configurations include star-shaped configurations (e.g., configurations in which three or more chains emanate from a single branch point), comb configurations (e.g., configurations having a main chain and a plurality of side chains, also referred to as “graft” or “bottlebrush” configurations), dendritic configurations (e.g., arborescent and hyperbranched polymers), mushroom side chains, and so forth. Thus, the biomimetic proteoglycan may have any shape, non-limiting examples of which include but is not limited to, cyclic, linear, branched, star-shaped, comb, graft, bottlebrush, dendritic, mushroom, and any combination thereof.

In certain embodiments, the reaction between the core polymer and the GAG is run at room temperature, or at a temperature from about 0° C. to about room temperature, or at a temperature from about room temperature to about the temperature where the reaction mixture boils. In other embodiments, the reaction is run with intense mixing, in order to promote constant contact between the aqueous solution and the organic solution. In yet other embodiment, the reaction is run for about 3 hours, or 6 hours, or about 9 hours, or about 12 hours, or about 15 hours, or about 18 hours, or about 21 hours, or about 24 hours, or about 30 hours, or about 36 hours, or about 42 hours, or about 48 hours, or any fractions or multiples thereof.

Reaction kinetics may be monitored using for example the fluorescamine assay. Fluorescamine (4′-phenylspiro[2-benzofuran-3,2′-furan]-1,3′-dione) is a spiro compound that is not fluorescent itself, but reacts with primary amines to form highly fluorescent products. When activated with the UV LED (365 nm), the fluorescence of the complex of fluorescamine with the primary amine bearing compound has an emission wavelength of approximately 490 nm.

The biomimetic proteoglycans prepared using the methods of the invention may be purified using, for example, dialysis, precipitation, extraction, chromatography (including affinity chromatography), and their chemical structures may be confirmed using spectroscopic methods, such as mass spectrometry and/or ¹H or ¹³C NMR spectroscopy. Macromolecular configuration of the biomimetic proteoglycans may be determined using AFM (atomic force microscopy). Cytocompatibility of the biomimetic proteoglycans may be assessed in cell cultures comprising, for example, fibroblasts. Osmotic pressure and water uptake of solutions of the biomimetic proteoglycans may be measured by gel osmometry and TGA (thermogravimetric analysis), respectively. In a non-limiting embodiment, binding affinity the biomimetic proteoglycans to collagen can be determined with the BioSensor chip.

In certain embodiments, the biomimetic proteoglycan comprises a GAG chain that is modified. For example, the GAG chain can be modified to incorporate other functional elements such as tags for visualization or peptides for cellular recognition.

In certain embodiments, the size of the biomimetic proteoglycan is controlled so that a desired size is generated. Chondrotin sulfate, keratan sulfate and other GAGs can migrate thereby limiting their use as compared with the biomimetic proteoglycan of the invention.

In certain embodiments, the biomimetic proteoglycan is arranged in the bottle-brush structure such that the electrostatically charged bristle molecules are in close proximity to one another. The close proximity of the charged bristles provides electrostatic repulsions and steric hindrances that assist the biomimetic proteoglycan in resisting force. This allows for two mechanisms of tissue restoration, an increased osmotic potential as well as mechanical function. In some instances, if the GAG chains are arranged in close proximity on the biomimetic proteoglycan, the GAG chains can produce electrostatic repulsions that can contribute to the mechanical resistance of the biomimetic proteoglycan.

In certain embodiments, the electrostatic repulsions between closely packed GAG chains generate a mechanical resistance to force, thereby restoring mechanical function to the tissue. Thus, the biomimetic proteoglycan can be generated to exhibit both a desirable mechanical property as well as a desirable osmotic pressure when placed into the tissue of a mammal in need thereof.

In addition to the ability to generate desired sizes of biomimetic proteoglycan, it is also possible according to the present invention to generate biomimetic proteoglycan that is variably susceptible to enzymatic digestion. In some instance, the biomimetic proteoglycan is susceptible to enzymatic digestion. In other instances, the biomimetic is resistant to enzymatic digestion.

Whether the proteoglycan is natural or biomimetic, the material of the invention can also be any combination of components making up a proteoglycan. For example, any combination of proteoglycan, HA, chondroitin sulfate, keratan sulfate, and the like can be administered into the tissue.

It will be understood from the present invention that other glycosaminoglycans and polysaccharides can be used for forming a biomimetic proteoglycan. For example, suitable glycosaminoglycans include HA, chondroitin, chondroitin sulfate, dermatan sulfate, heparan sulfate, keratan sulfate and heparin. In addition, any polymer that resembles a glycosaminoglycan can be used to generate the biomimetic proteoglycan of the invention. Based on the disclosure presented herein, a skilled artisan would understand that any hydrophilic polymer can be used.

The GAG chains and/or components thereof can be prepared using any method disclosed herein. For example, the materials can be isolated from a healthy donor. Preferably, the supply of the GAG chains and/or components thereof can be derived from a mammal, preferably a human. The GAG chains and/or components thereof can be autologous, allogenic, or xenogenic with respect to the recipient. Alternatively, the materials can be produced by a cell. In another aspect, the materials can be produced synthetically.

The invention is applicable to produce any biomimetic proteoglycan, such as aggrecan. As a non-limiting example, versican is a large proteoglycan of about 265 KDa with 12-15 chondroitin sulfate chains attached. This protein is a major component of the dermal layer of skin, and interacts with hyaluronan in the extracellular matrix through N-terminal contacts. Versican also interacts with numerous other signaling molecules through C-terminal contacts. The central domain of versican contains the glycosaminoglycan attachment points, but differential splicing in various tissues leads to a variety of glycosaninoglycan attachments and sulfation patterns, further yielding an assortment of glycosaminoglycan chain interactions with other molecules. In addition, since versican is known to interact with hyaluronan, increased versican production may increase hyaluronan production.

In addition to versican, dermis contains several small leucine-rich proteoglycans (SLRPs) such as decorin, biglycan and lumican. SLRPs plays an important role in the regulation of cell activity and in the organization and functional properties of skin connective tissue. A modification of their repartition might be involved in the alterations which occur in skin aging. It was shown that lumican expression decreased during aging whereas decorin expression tended to increase, resulting in a strong alteration of the decorin to lumican ratio.

Lumican has a 38 KDa protein core that contains two keratan sulfate GAG attachment sites, and has been shown to affect the integrity of the extracellular matrix and skin structure. For instance, knockout mice that could not express lumican displayed abnormal collagen assembly and brittle skin, suggesting lumican plays a large role in extracellular matrix (ECM) maintenance and in skin health (Wegrowski, et al., Mol Cell Biochem 205(1-2): 125-31, 2000; Vuillermoz, et al., Mol Cell Biochem 277(1-2): 63-72, 2005). Periodontal health is also affected by lumican removal due to its interactions with collagen (Matheson, et al., J Periodontal Res 40(4): 312-24, 2005). In addition, Roughley et al., 1996, Biochem J. 318:779 indicated a role for lumican and other SLRPs in protecting collagen from degradation by collagenases, further suggesting a role for lumican in ECM maintenance and prevention of ECM degradation (Geng, et al., 2006, Matrix Biol., 25(8):484-91). Further, Vuillermoz et al. showed that lumican expression decreased in skin fibroblasts with increased age, suggesting a possible role of lumican in age-related damage to skin. In addition, lumican plays a role in corneal health, as decreased or knocked-out lumican expression resulted in poor corneal formation (Chakravarti, Glycoconj J 19(4-5): 287-93, 2002), further supporting a role in collagen fibril formation, but, also, purified lumican has been shown to promote corneal epithelial wound healing (Yeh, et al. Opthalmol Vis Sci 46(2): 479-86, 2005). Therefore, delivery of biomimetic lumican to skin may facilitate collagen fibril formation and increase the water content due to the charge and hydrophilicity of the glycosaminoglycan chains, thereby increasing skin health and appearance.

Other known proteoglycans include syndecans 1-4, glypicans 1-5, betaglycan, NG2/CSPG4, CD44/epican, fibromodulin, PRELP, keratocan, osteoadherin/osteomodulin, epiphycan, osteoglycin/mimecan, neurocan/CSPG3, brevican, bamacan, agrin, and serglycin.

### Grafting

The methods of generating a biomimetic proteoglycan discussed elsewhere herein are applicable to general grafting methodologies. Grafting copolymers contain side-chain branches emanating from different points along the polymer backbone. Variations in the nature of the main chain and side chains, in the length and polydispersity of the backbone and branches as well as in graft density determine the properties of the resulting graft copolymer. These variables also relate to the synthetic complexity of preparing these copolymers.

Graft copolymers can generally be prepared by the “onto”, “through” and “from” grafting processes. In the “grafting onto” process, end-functionalized polymer chains are attached to the main chain of another polymer by coupling reactions with functional groups along its backbone. “Grafting onto” is used herein interchangeably with “grafting to”.

In certain embodiments, the biomimetic proteoglycan can be fabricated via the “grafting to” method wherein a GAG chain is grafted to a functional polymer.

In certain embodiments, the biomimetic proteoglycan can be fabricated via the “grafting from” method wherein a disaccharide unit of a GAG chain (e.g., GlcUA and GalNAc) is attached to a polymeric backbone. Subsequent disaccharide or saccharide units are then grown from the polymeric backbone using enzymes of GAG synthesis such as but not limited to GlcA I transferase, GlaNAc transferase, chondroitin synthase, chondroitin 6-0 sulfotransferase and chondroitin 4-O-sulfotransferase.

In certain embodiments, the biomimetic proteoglycan prepared using any of the grafting methods disclosed elsewhere herein is end-functionalized with, but not limited to, a hyaluronan binding region or collagen binding region. Polymerizations that can be used to join into a backbone multiple terminal ends of biomimetic proteoglycan bottle brushes can include but are not limited to radical polymerization, cationic polymerization, living anionic polymerization, atom transfer radical polymerization, and ring opening metathesis polymerization.

Treatment of Soft Tissue Defects and Disorders

The compositions disclosed herein may be used to treat any number of soft tissue disorders and defects. For example, functional restoration of cartilage and/or the meniscus in the knee may be accomplished by administering the compositions of the invention to the knee. Similarly, soft tissue disorders and defects in other body tissues, including, but not limited to skin, heart valve, articular cartilage, fatty tissue, craniofacial, ocular, tendon, ligament, fascia, fibrous tissue, synovial membrane, muscle, nerves, intervertebral disc, urethra, bone and blood vessel. Disorders or defects in any one of these sites may be treated by administering the compositions of the invention to the respective site. Thus, the invention should be construed to include treatment of soft tissue defects and disorders to effect functional restoration of the same. The precise methods to be used will be readily apparent to the skilled artisan with experience in the soft tissue in question.

The present invention also provides methods for soft tissue restoration and/or augmentation in a subject, the methods comprising administering a composition of the present invention to a mammal in need thereof. The method of the invention is designed to improve conditions including, but not limited to, lines, folds, wrinkles, minor facial depressions, cleft lips, correction of minor deformities due to aging or disease, deformities of the vocal cords or glottis, deformities of the lip, crow's feet and the orbital groove around the eye, breast deformities, chin deformities, cheek and/or nose deformities, acne, surgical scars, scars due to radiation damage or trauma scars, and rhytids. The soft tissue can also be located in the pelvic floor, in the peri-urethral area, near the neck of the urinary bladder, or at the junction of the urinary bladder and the ureter. The method of soft tissue augmentation may increase tissue volume. The compositions may be administered into the skin or may be administered underneath the skin. The compositions include insoluble elastin derived from human vascular tissue that does not induce inflammatory or immune response and does not induce calcification.

The term “soft tissue augmentation” includes, but is not limited to, the following: dermal tissue augmentation; filling of lines, folds, wrinkles, minor facial depressions, cleft lips and the like, especially in the face and neck; correction of minor deformities due to aging or disease, including in the hands and feet, fingers and toes; augmentation of the vocal cords or glottis to rehabilitate speech; hemostatic agent, dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; bulking agent for periurethral support, filling of indentations in the soft tissue, dermal or subcutaneous, due to, e.g., overzealous liposuction or other trauma; filling of acne or traumatic scars and rhytids; filling of nasolabial lines, nasoglabellar lines and infraoral lines.

The term “augmentation” means the repair, decrease, reduction or alleviation of at least one symptom or defect attributed due to loss or absence of tissue, by providing, supplying, augmenting, or replacing such tissue with the composition of the present invention. The compositions of the present invention can also be used to prevent at least one symptom or defect in the tissue.

### Administration

The compositions of the present invention may be administered to a soft tissue site in a mammal, for the functional restoration thereof, using a variety of methods and in a variety of formulations known in the art. The mammal is preferably a human.

In some instances, the composition of the invention does not appreciably degrade following administration. In other instances, the composition of the invention degrades either rapidly, or slowly, in the tissue. Thus, when administered in the body, a biomimetic proteoglycan, such as biomimetic aggrecan, may be permanent, may be degraded enzymatically, or may be degraded in the presence of a solvent, such as, for example, water.

To facilitate administration, the proteoglycan can be delivered to the mammal in a carrier. The carrier can be water or another liquid in which proteoglycan is soluble. Likewise the liquid can be one in which the proteoglycan does not dissolve, such as a biocompatible oil. The concentration of proteoglycan in the carrier can be such that that the volume of material administered, carrier and proteoglycan, either swells or contracts in vivo. The idea is to administer a specific amount of proteoglycan sufficient to restore function of the tissue. Preferably, the material swells in vivo. This means that the proteoglycan concentration must be below its capacity to absorb and hold water in the nucleus environment. It is believed that such a concentration would not be flowable. In such case, the non-solvent carrier can be used. The non-solvent carrier can migrate out of the tissue and allow the proteoglycan to swell. If the required concentration was not flowable, a fraction of the desired concentration can be used and administered into the tissue of successive days or weeks to build the desired concentration in the tissue, for example, one third concentration would require three administrations. The method may include a single administration or a series of administrations in order obtain desired tissue restoration.

Without wishing to be bound by any particular theory, the biomimetic proteoglycan solution may contain a clinically relevant amount of biomimetic proteoglycan. This clinically relevant amount can be determined by measuring the concentration of proteoglycan in normal tissue and in degenerated tissue. The difference between these two measurements would theoretically be the amount needed. This amount should be available in a volume that could be administered in a tissue. The proteoglycan can be administered with no tissue preparation, or some material from the existing tissue can be removed to make appropriate room for the biomimetic proteoglycan. The biomimetic proteoglycan can also be packaged as a dry substance that can be reconstituted prior to use.

Another method of accomplishing the same goal of restoring the load carrying capability of the tissue includes administering biomimetic proteoglycan, HA, chondroitin and keratan sulfate and allowing the components to self-aggregate to form proteoglycan in the tissue in vivo. These components can also be modified with proteins to facilitate their self-agglomeration. The components can be xenograft, allograft or synthetic, or analogs thereof.

The compositions of the present invention can take the form of immediate release (injection) formulations, or delayed release formulations, i.e., using microspheres, nanospheres or other matrices such as hydrogels for controlled release.

The present invention provides a method for restoring a damaged or degenerated tissue, comprising administering an administrable formulation comprising the biomimetic proteoglycan (and/or components thereof). The administrable formulation can either be viscous or form a solid or gel in situ.

In another embodiment of the present invention, the administrable formulation is an aqueous solution. In a specific embodiment, the administrable formulation comprises an aqueous solution containing a biopolymer such as a cellulosic, a polypeptidic or a polysaccharide or a mixture thereof. One specific biopolymer is chitosan, a natural partially N-deacetylated poly(N-acetyl-D-glucosamine) derived from marine chitin. Other specific biopolymers include collagen (of various types and origins). Other biopolymers of interest include methyl cellulose, hydroxyethyl cellulose, hydroxypropyl methyl cellulose, hyaluronic acid, and the like.

In specific embodiments of this invention, the administrable formulation preferably comprises an aqueous solution containing a water-soluble dibasic phosphate salt. The administrable formulation may contain a mixture of different water-soluble dibasic phosphate salts. Contemplated dibasic phosphate salts comprise dibasic sodium and magnesium mono-phosphate salts as well as monophosphate salt of a poly or sugar. This does not exclude the use of water-soluble dibasic salts other than phosphate, such as carboxylate, sulfate, sulfonate, and the like. Other formulations of the method may contain hyaluronic acid or chondroitin sulfate or synthetic polymers such poly(ethylene glycol) or poly(propylene glycol), and the like.

In other embodiments of this invention, the administrable in situ setting formulation is nonaqueous (does not contain water) and solid or gel forming (turns into a solid or gel in situ).

In another embodiment of this invention, the administrable formulation is nonaqueous and comprises an organic solvent or a mixture of organic solvents. Metabolically absorbable solvents are preferably selected (triacetin, ethyl acetate, ethyl laurate, and the like).

In another embodiment of this invention, the administrable formulation is nonaqueous and contains at least one fatty acid or a mixture of fatty acids. The administrable formulation comprises saturated or unsaturated fatty acid selected from the group consisting of palmitate, stearate, myristate, palmitoleate, oleate, vaccenate and linoleate. It may be a mixture of several of these fatty acids. The fatty acid may be mixed with a metabolically absorbable solvent or liquid vehicle to reduce viscosity and allow administrability.

In yet another embodiment, the administrable formulation is a dry powder, which when introduced into the tissue is hydrated within the tissue to result in the desired restoration thereof.

In the method of the present invention, a low viscosity formulation is administered into tissue. The formulation is administered easily, with a minimal pressure, through the fine tube of a needle or catheter. Typical tube gauge ranges are from 13 to 27. In another embodiment, the biomimetic proteoglycans could be administered via microneedles. Administrations are performed by instruments or devices that provide an appropriate positive pressure, e.g. hand-pressure, mechanical pressure, injection gun, etc. One representative technique is to use a hypodermic syringe.

The invention also includes a method of administering the biomimetic proteoglycans and/or components thereof by way of simple injection through a needle preferably 18 gauge or smaller or a small cannula, preferably 2 mm or less in diameter. A contemplated administration site is at or around the tissue. It is envisioned that the biomimetic proteoglycans and/or components thereof can be pre-packaged sterilely in syringes for easy and safe use.

### Combination Therapy

The biomimetic proteoglycan can be administered to a mammal in need therefore alone or in combination with additional components including but not limited to hyaluronic acid, a hyaluronic acid analog, elastin or collagen.

In one embodiment, the biomimetic proteoglycan can be combined with a biomolecule (such as a nucleic acid, amino acid, sugar or lipid). Such a biomolecule can be covalently attached or non-covalently associated with the biomimetic proteoglycan described herein. In an exemplary embodiment, the biomolecule is selected from a group consisting of a receptor molecule, extracellular matrix component and a biochemical factor. In another exemplary embodiment, the biochemical factor is a growth factor and/or a differentiation factor.

In another exemplary embodiment, the biomimetic proteoglycan of the invention can be combined with a first molecule (which may or may not be a biomolecule). Such a first molecule can be covalently attached to the biomimetic proteoglycan of the invention. This first molecule can be used to also interact with a biomolecule discussed above. In an exemplary embodiment, the first molecule is a linker, and the second biomolecule is a member selected from the group consisting of a receptor molecule, biochemical factor, growth factor and a differentiation factor. In an exemplary embodiment, the first molecule is selected from the group consisting of heparin, heparan sulfate, heparan sulfate proteoglycan, and combinations thereof. In an exemplary embodiment, the second biomolecule is selected from the group consisting of a receptor molecule, biochemical factor, growth factor and a differentiation factor. In another exemplary embodiment, the first molecule is covalently attached through a linker, and said linker is selected from the group consisting of di-amino poly(ethylene glycol), poly(ethylene glycol) and combinations thereof. For biomolecules that do not bind to heparin, direct conjugation to the polymer scaffold or through a linker (such as PEG, amino-PEG and di-amino-PEG) is also feasible. In another exemplary embodiment, the biomolecule is an extracellular matrix component that is selected from the group consisting of laminin, collagen, fibronectin, elastin, vitronectin, fibrinogen, polylysine, other cell adhesion promoting polypeptides and combinations thereof. In another exemplary embodiment, the biomolecule is a growth factor selected from the group consisting of acidic fibroblast growth factor, basic fibroblast growth factor, nerve growth factor, brain-derived neurotrophic factor, insulin-like growth factor, platelet derived growth factor, transforming growth factor beta, vascular endothelial growth factor, epidermal growth factor, keratanocyte growth factor and combinations thereof. In another exemplary embodiment, the biomolecule is a differentiation factor selected from the group consisting of stromal cell derived factor, sonic hedgehog, bone morphogenic proteins, notch ligands, Wnt and combinations thereof.

The first molecules covalently attached to the biomimetic proteoglycan of the invention can be used to interact with a biomolecule (for example, a growth factor and/or ECM component) in order to stimulate cell growth. In another exemplary embodiment, the biomimetic proteoglycan can be used for wound healing, and the biomolecule is selected from the group consisting of an extracellular matrix component, growth factors and differentiation factors. Examples of potential factors for wound healing enhancement include epidermal growth factor (EGF), vascular endothelial growth factor (VEGF), basic fibroblast growth factor (bFGF) and platelet-derived growth factor (PDGF).

Biomolecules can be incorporated within the compositions of the invention during fabrication or post-fabrication. These biomolecules can be incorporated via covalent attachment directly or through various linkers or by adsorption.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations which are evident as a result of the teachings provided herein.

### Materials and Methods

Chondroitin sulfate (CS), natural aggrecan, 1× phosphate buffer saline (PBS), fluorescamine, ethyl acetate (EtOAc), sodium borate buffer (SBB), dimethyl sulfoxide (DMSO), 3-aminopropyltriethoxysilane (APTES), deuterium oxide and poly(ethylene glycol) were purchased from Sigma-Aldrich, Saint Louis.

Poly(acryloyl chloride) (PAC) (MW 10 kDa, 25% solution in dioxane) was purchased from Polysciences, Warrington Pa.

Dialysis membranes (RC6, MWCO 50 kDa and MWCO 1,000 Da) were purchased from Spectrum Labs, Rancho Dominguez, Calif.

Sephadex G-50 (GE Healthcare) desalting columns were obtained from Fisher Scientific, Pittsburgh, Pa.

Hyaluronic acid sodium salt (HA) (MW 2,000 kDa) was obtained from Lifecore Biomedical, LLC, Chaska, Minn.

### Fluorescamine Assay:

Change in concentration of primary amines is determined with the fluorescamine assay. 50 μL of the 10 mM fluorescamine solution in DMSO are added to 150 μL of the tested biomimetic proteoglycan solution in a 96 well plate (Corning). The solution is allowed to mix for 5 min while on shaker plate. Fluorescence intensity is read using Tecan 2000 spectrophotometer (excitation 365 nm, emission 490 nm, gain 80). Each solution is tested at least in triplicate. Conjugation of CS to a polymer backbone is determined by comparing signal intensity of biomimetic proteoglycan samples to non-reacted CS solutions of the same concentration.

### Dialysis and Lyophilization:

After 24 hrs of reaction, samples are dialyzed using CR6 membrane (MWCO 50 kDa) against DI water for 96 hrs. DI water is changed every 24 hr. Following dialysis, samples are frozen and lyophilized (Labconco, FreeZone benchtop system) at −90° C., 0.189 mBarr. Purified samples are reconstituted at desired concentrations and used for further characterization.

### ¹H NMR:

25 mg/ml lyophilized samples are solubilized in deuterium oxide (D₂O). ¹H NMR spectra are taken on a 300 MHz NMR spectrometer (UNITYNOVA, Mckinley Scientific, Sparta, N.J.) at 64 scans and at ambient temperature.

### AFM:

Cleaved mica surface is functionalized with 50 μL of 0.03% APTES solution in a closed container by incubation for 30 minutes at a room temperature. Following incubation, it is rinsed gently in a stream of MiliQ water and air dried. 50 μL of 25 μL/ml of natural aggrecan and biomimetic proteoglycan solutions are incubated on a functionalized mica surface for 15 minutes, samples are rinsed gently in a stream of MiliQ water and dried. AFM images are obtained with Digital Instruments Nanoscope.

### Cytotoxicity:

L929 fibroblasts are seeded at a density 13,000 cell/m² on 12 well tissue culture plates (RPMI media, 5% fetal bovine serum, L-glutamine and 1% pen/strep) and allowed to attach for 24 hrs before dosing with 0.2 mg/ml and 2 mg/ml solutions of CS and biomimetic proteoglycan (sterilized via exposure to UV light for 2 hrs). Cell viability is investigated after 48 hrs using LIVE/DEAD Viability Cytotoxicity Kit (Invitrogen). Images are collected on an inverted fluorescent microscope and processed with Image J software.

### Osmotic Pressure:

Swelling degree of Sephadex gel beads G-50 (15-50 μm dia) is calibrated using poly(ethylene glycol) solutions (1×PBS) of known osmotic pressure (5-100 kPa) using gel osmometry technique. Briefly, a single bead is isolated on a glass slide, a drop of solution is added, and change in a bead volume is monitored via a microscope camera. Images corresponding to a dry state and equilibrated swollen state are analyzed with Image J software, and a swelling degree is calculated based on a change in a radial dimension of a sphere under an assumption of uniform swelling.

Osmotic pressure of CS, natural aggrecan and biomimetic proteoglycan at different concentrations is determined from a calibration curve for Sephadex G-50, after swelling gel beads in respective solutions.

### Water Uptake:

Water uptake measurements are performed on CS, natural aggrecan, hyaluronic acid and biomimetic proteoglycan using Thermal Gravimetric Analyzer at 90% relative humidity, 37° C. over 24 hrs after drying samples in a vacuum oven at 37° C. for three days.

### Binding Assay

Binding affinity of natural aggrecan and biomimetic proteoglycan to collagen type II was determined with the BioSensor chip, when proteoglycan solutions of different concentrations were injected onto sensor surfaces on which collagen was immobilized. Change in the surface resonance upon binding of proteoglycans to collagen II was detected optically and measured in resonance units.

### Example 1

For reaction with CS, 0.105 mM solution of PAC in EtOAc was prepared by directly dissolving PAC/dioxane mixture in EtOAc. In particular, a 11.4 mM solution of PAC in EtOAc was prepared by adding 126 μL of PAC (25% solution in dioxane) to 30 ml of EtOAc. M (PAC) [molarity of PAC]=0.126×0.25/(0.03×10,000)=0.105 mM; M (AC) [molarity in acyl chloride]=0.126 g×0.25/(0.03 L×92 g/mol)=11.4 mM. M (AC) is equal to M(PAC) multiplied by numbers of AC monomers in PAC, that is 0.105 mM multiplied by M_{w}(PAC)/M_{w}(AC) (which corresponds to 10,000 g/mol/92 g/mol).

CS solution (1.14 mM, 25 mg/ml) was prepared in SBB (0.1 M, pH 9.4). In particular, a solution of CS (1.14 mM) in SBB was prepared by dissolving 1.050 g of CS in SBB to a final volume of 42 mL. The molarity of the solution in terms of CS was 1.050 g/(0.042 L×22,000 g/mol)=1.14 mM.

Equal volumes of PAC and CS solutions were combined to provide ˜1CS:10 acyl chlorides (mol) ratio. For example, 10 ml of 11.4 mM PAC solution were mixed with 10 ml of CS solution in SBB.

As controls, a mixture of 10 ml of EtOAc with 10 ml of CS solution in SBB was used as control 2, and 2 ml of CS solution in SBB was used as control 1.

The two-phase system was vigorously stirred for 10 minutes, and then allowed to react for 24 hrs mixed continuously (Thermolyne Labquake).

As a non-limiting example, a bottle-brush, chondroitin sulfate-containing, biosynthetic proteoglycan prepared using the methods of the invention showed 3-5 nm bristle spacing and about 9 bristles attached onto PAC core of MW of about 10 kDa. By comparison, natural aggrecan has ˜100 bristles spaced at 2-3 nm and a MW of ˜2,000-3,000 kDa.

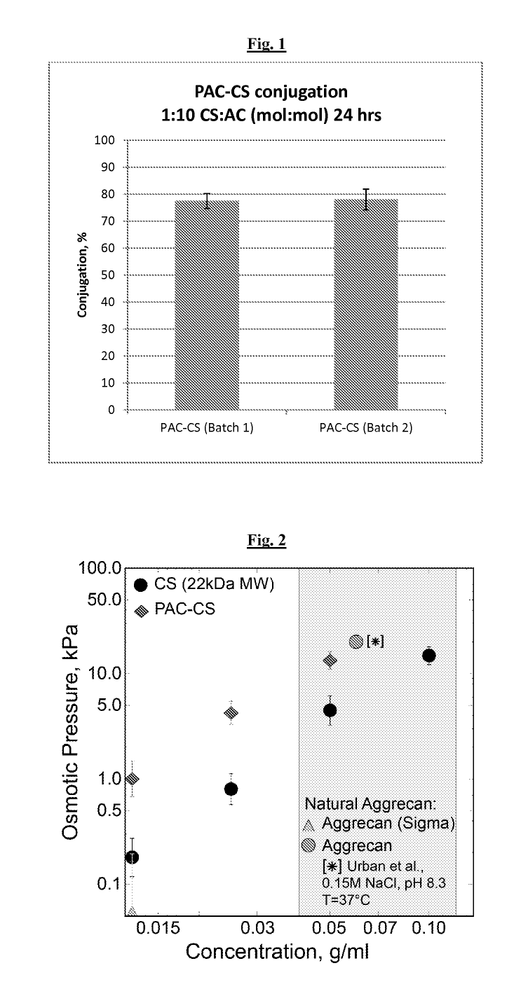

FIG. 1 illustrates conjugation results obtained with the methods of the invention.

¹H-NMR showed characteristic peaks of CS and a polymer backbone, confirming no degradation of CS bristles after synthesis and purification.

The results of the cytotoxicity study showed that biomimetic aggrecan did not affect cell viability as compared to non-treated controls of CS at similar concentrations.

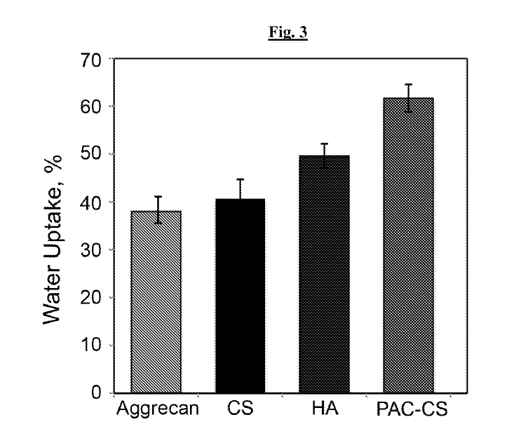

Osmotic pressure of solution of PAC-CS in 1×PBS (pH 7.4) ranged from ˜4 kPa at 25 mg/ml to ˜14 kPa at 50 mg/ml, was statistically higher than osmotic pressure of CS alone (p<0.001 at 50 mg/ml), and was in the range of osmotic pressure of natural aggrecan (FIG. 2). PAC-CS had a water uptake of 63%, compared to 43% for CS alone, perhaps due to the charges along the polymer backbone (FIG. 3). The biosensor results indicated that natural aggrecan and biomimetic proteoglycan have similar binding affinities to collagen type II with the dissociation constant K_{D} in the range of 10⁻⁴ M.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of preparing a biomimetic proteoglycan, the method comprising contacting a core polymer comprising at least one acyl chloride group and a GAG comprising a terminal primary amine, thereby forming a biomimetic proteoglycan.

2. The method of claim 1, wherein the GAG is selected from the group consisting of hyaluronic acid, chondroitin, chondroitin sulfate, heparin, heparin sulfate, dermatan, dermatan sulfate, laminin, keratan sulfate, chitin, chitosan, acetyl-glucosamine, oligosaccharides, and any combinations thereof.

3. The method of claim 2, wherein the GAG comprises chondroitin sulfate.

4. The method of claim 1, wherein the core polymer is in an organic solution, wherein the organic solution is not fully soluble in water or an aqueous solution.

5. The method of claim 4, wherein the core polymer is in an organic solution comprising ethyl acetate, dioxane, tetrahydrofuran, dichloroethane, dichloromethane, cyclohexane, or any mixtures thereof.

6. The method of claim 1, wherein the GAG is in an aqueous solution.

7. The method of claim 6, wherein the GAG is in a buffered aqueous solution.

8. The method of claim 7, wherein the GAG is in an aqueous solution of pH ranging from about 5.5 to about 9.4.

9. The method of claim 1, wherein the polymer core comprises acyl chloride-containing derivatives of poly(acrylic acid), poly(methacrylic acid), poly(glutamic acid) or poly(aspartic acid), or copolymers, mixtures, and combinations thereof.

10. The method of claim 1, wherein at least a portion of the acyl chloride groups in the core polymer does not react with the terminal primary amines of the GAGs.

11. The method of claim 10, further comprising reacting the unreacted acyl chloride groups with a nucleophile or base.

12. The method of claim 10, wherein all or about all of the acyl chloride groups in the core polymer react with the terminal amines of the GAGs.

13. The method of claim 1, wherein the biomimetic proteoglycan is resistant to enzymatic breakdown in a mammalian in vivo environment.

14. The method of claim 1, wherein the biomimetic proteoglycan has a shape selected from the group consisting of cyclic, linear, branched, star-shaped, comb, graft, bottlebrush, dendritic, mushroom, and any combinations thereof.

15. The method of claim 1, wherein the biomimetic proteoglycan mimics a natural proteoglycan selected from the group consisting of aggrecan, betaglycan, decorin, perlecan, serglycin, syndecan-1, biglycan, fibromodulin, lumican, versican, neurocan, brevican, and any combinations thereof.

16. A composition comprising a biomimetic proteoglycan prepared according to the method of claim 1.

17. The composition of claim 16, further comprising at least one biologically active molecule selected from the group consisting of a growth factor, cytokine, antibiotic, protein, anti-inflammatory agent, and analgesic.

18. A method of treating a disease, disorder, or condition associated with a soft tissue in a mammal in need thereof, the method comprising administering to the mammal a therapeutically effective amount of at least one composition of claim 16.

19. The method of claim 18, wherein the soft tissue is selected from the group consisting of intervertebral disc, skin, heart valve, articular cartilage, cartilage, meniscus, fatty tissue, craniofacial, ocular, tendon, ligament, fascia, fibrous tissue, urethra, bone, synovial membrane, muscle, nerves, blood vessel, and any combinations thereof.

20. The method of claim 18, wherein the biomimetic proteoglycan mimics a natural proteoglycan selected from the group consisting of aggrecan, betaglycan, decorin, perlecan, serglycin, syndecan-1, biglycan, fibromodulin, lumican, versican, neurocan, brevican, and any combinations thereof.

21. (canceled)
